(19) United States
(12) Patent Application Publication
(10) Pub. No.: US 2008/0207644 A1
(43) Pub. Date: Aug. 28, 2008

(54) Therapeutic materials and methods

(76) Inventors: Stephen T. Sonis, Wayland, MA (US); Camille L. Bedrosian, Belmont, MA (US)

Correspondence Address: ARIAD PHARMACEUTICALS, INC.;ARIAD GENE THERAPEUTICS, INC., 26 LANDSDOWNE ST., CAMBRIDGE, MA, 02139, US

(21) Appl. No.: 11/986,878

(22) Filed: Nov. 27, 2007

Related U.S. Application Data
(60) Provisional application No. 60/861,123, filed on Nov. 27, 2006.

(51) Int. Cl. A61K 31/522; A61K 31/4965; A61K 31/454; A61P 35/00

(52) U.S. Cl. 514/255.06; 514/263.32; 514/323

(57) ABSTRACT

Disclosed are methods for treating various cancers. Methods encompass the administration of an mTOR inhibitor in combination with a second drug selected from an ImiD, a PDE4 inhibitor, a p38 MAP kinase inhibitor, a xanthine anticytokine, a dual TACE/MMP inhibitor and a proteasome inhibitor.

The methods are aimed at providing a desirable therapeutic window while maintaining prior, if not higher, dose levels of the mTOR inhibitor.

### TECHNICAL FIELD

This invention provides compositions and methods for treating cancer.

### BACKGROUND

Several mTOR inhibitors are currently under evaluation as single agents or in various combinations for the treatment of a variety of cancers. Those mTOR inhibitors include the rapamycin analogs, AP23573 (ARIAD Pharmaceuticals, Inc.), everolimus (Novartis) and temsirolimus (Wyeth). Other mTOR inhibitors include, among others, sirolimus (rapamycin), and the additional analogs, ABT-578 and biolimus. While AP23573, everolimus and temsirolimus have all yielded positive results in human studies, mouth sores have been noted as a dose limiting toxicity.

Those mouth sores have previously been loosely termed “mucositis” in some cases. Actually, however, they typically differ noticeably from the classic mucositis that frequently accompanies radiation therapy and other cancer therapies such as cytotoxic cancer chemotherapies. Nonetheless, these mouth sores can be debilitating and constitute a dose limiting toxicity for the use of the new mTOR inhibitors.

One approach sometimes used in order to reduce drug doses and thus to alleviate side effects is to shift from monotherapy to combination therapy, where the addition of a second drug permits smaller doses of the first drug, and thus, reduced incidence or severity of side effects. This invention takes a somewhat different approach.

### SUMMARY OF THE INVENTION

We now provide an mTOR inhibitor combination therapy for achieving a desirable therapeutic window preferably while maintaining prior, if not higher, dose levels of the mTOR inhibitor.

The mTOR inhibitor may be rapamycin or a rapamycin analog, and may be administered by any pharmaceutically acceptable route, a variety of which are known for that class of drugs. Oral or parenteral administration is currently of particular interest. The mTOR inhibitors of greatest current interest are rapamycin analogs in which the hydroxyl group at position 43 is replaced, especially those analogs currently in clinical development for treating cancer, such as AP23573, Everolimus and Temsirolimus. These and other mTOR inhibitors are discussed in greater detail below.

Dose levels for the mTOR inhibitor in this combination therapy are generally in the range of 25-800 mg/week, e.g., in some cases 100-500 mg/week. Such weekly dosage levels may be achieved for example with daily oral administration of 5-160 mg, provided up to 7 days per week (e.g., 30-100 mg QDx6; i.e. administration of 30-100 mg/day for 6 consecutive days), or with i.v. infusions one or more times per 7-21-day period. Of particular interest are dose levels and dosing schedules at least as high/frequent as those approved or under study for rapamycin or its analogs, whether in a monotherapy or drug combination regimen. In dosing schedules involving administration of the rapamycin analog for 3 or more days per week, e.g., every day, QDx4, QDx5, or QDx6 (i.e. administration of the rapamycin analog for 4, 5 or 6 consecutive days), doses of 20 mg and greater are of special interest, and are of increasing interest at or above levels of 30 mg, 40 mg, 50 mg, and 60 mg and higher. The dosing schedule may be intermittent. “Intermittent” dosing refers to schedules providing intervening periods between doses, e.g. every second day dosing, every third day dosing, or more generally, schedules containing “holidays” of one or more days or weeks between periods of dosing. A non-limiting example of such intermittent dosing is a cycle of one week of QDx4 (or QDx5 or QDx6) dosing followed by one week (or two or three weeks) without drug, then resuming with another week of drug treatment followed by a week (or weeks) without drug treatment, and so on. To illustrate further, administration of 60 mg QDx6 every other week provides a weekly dose of 360 mg of drug on an intermittent basis (i.e., every other week).

A second drug, a “small molecule”, i.e., typically having a molecular weight less than 750 atomic mass units, usually less than 550 amu, is administered in conjunction with the mTOR inhibitor. As discussed in greater detail below, the second drug may be an IMiD (such as thalidomide, Revlimid (lenalidomide) or Actimid (CC-4047)), a PDE4 inhibitor (such as SB207499, CAS Registry No 153259-65-5), a p38 MAP kinase inhibitor (such as BIRB 796, SCIO-469 or VX-702 (CAS Registry No. 479543-46-9)), a xanthine anticytokine (such as pentoxiphylline), a dual TACE/MMP inhibitor (such as TMI 1, CAS Registry No 287403-39-8), a proteasome inhibitor (such as Velcade) or a kinase inhibitor for IKK (such as BMS 345541, CAS Registry No 547757-23-3).

The second drugs disclosed herein have generally been studied and/or used as orally or parenterally administered products, and a significant literature has been established in connection with such use. In the present case, however, embodiments of greatest current interest involve administering the second drug in a formulation suitable for mucosal delivery. This generally involves a liquid, gel or otherwise fluid composition, typically containing 0.1-10% (by weight) of the second drug, for use as a mouthwash, mouth rinse, gargle, liquid bandage or other form suitable for providing local or topical contact with the oral mucosa (i.e., the lining of the oral cavity). A typical dose of interest is 0.3 to 5 times the dose levels approved or studied for the respective second drug when given systemically or orally.

The rapamycin or rapamycin analog is co-administered to the patient with one of the second drugs, i.e., the two drugs are administered to the patient simultaneously or sequentially, in any given order and by the same or different routes of administration. It is of particular current interest to initiate administration of the second drug first, for example, one or more days or weeks prior to initiation of administration of the mTOR inhibitor.

Given the documented activity of mTOR inhibitors against a wide variety of cancers, the combination therapy disclosed herein should be of interest for a correspondingly wide range of cancers. Those include among others prostate, endometrial, breast, ovarian, cervical, head and neck, small cell and non-small cell lung, pancreatic, kidney, brain, colorectal and bladder cancers as well as various sarcomas (including the various bone and soft tissue sarcomas), melanomas, multiple myeloma, B-cell lymphoma, mantle cell lymphoma, Non-Hodgkin's Lymphoma, CLL and CML, including, among others, cases which are advanced, recurrent, refractory to one or more other therapies and/or metastatic.

Moreover, additional drugs such as those described below may be given in conjunction with the combination therapy of this invention. As disclosed herein, this invention thus provides the use of such a second drug for the preparation of a medicament for the treatment of a cancer patient who is or will also be treated with an mTOR inhibitor.

### DETAILED DESCRIPTION

The combination therapy disclosed herein constitutes a new method for treating various types of cancer, with the objective of providing a desirable therapeutic window for achieving clinical benefit without incurring an unacceptable level of side effects.

As used herein, the term “treating” refers to the administration of an mTOR inhibitor and a second drug to a patient after the onset, or suspected onset, of a cancer. “Treating” includes the concepts of “alleviating”, which refers to lessening the frequency of occurrence or recurrence, or the severity, of any symptoms or other ill effects related to a cancer and/or the side effects associated with cancer therapy. The term “treating” also encompasses the concept of “managing” which refers to reducing the severity of a particular disease or disorder in a patient or delaying its recurrence, e.g., lengthening the period of remission in a patient who had suffered from the disease.

The term “effective amount” or “effective dose”, as used herein means the amount or dose of a substance that elicits a desirable biological or clinical response in a tissue or patient. For example, a desirable response may include one or more of the following: delaying or preventing the onset of a medical condition, disease or disorder; slowing down or stopping the progression, aggravation or worsening of the condition or symptoms of the condition; ameliorating the symptoms of the condition; and curing the condition—e.g., where the condition, disease, disorder or symptoms is, or is associated with cancer or with side effects of anti cancer therapy.

All combinations of method or process steps as used herein can be performed in any order, unless otherwise specified or clearly implied to the contrary by the context in which the referenced combination of steps is made.

All percentages, parts and ratios as used herein are by weight of the total composition, unless otherwise specified. All such weights as they pertain to listed ingredients are based on each respective ingredient per se and, therefore; do not include solvents or by-products that may be included in commercially available materials, unless otherwise specified.

The compositions and methods of the present invention can comprise additional or optional ingredients, components, or limitations described herein or otherwise useful in compositions and methods of the general type as described herein.

1. Rapamycin Analogs—mTOR Inhibitors

Rapamycin is a macrolide produced by Streptomyces hygroscopicus and discovered in the 1970's. Rapamycin is a potent immunosuppressive agent and is used clinically to prevent rejection of transplanted organs. It has also been reported to have a wide range of interesting pharmacologic activities, including certain anti-cancer activity. See e.g. US Pat. appln 2001/0010920. A number of derivatives of rapamycin, including AP23573 (ARIAD), CCI779 (“temsirolimus”, Wyeth) and RAD001 (“Everolimus”, Novartis) have yielded promising results in human studies against a variety of cancers. In addition, rapamycin and everolimus are used as immunosuppressants in organ transplant recipients. Rapamycin and a number of the C-43-modified rapamycin analogs, including among others AP23573, Biolimus and ABT-578 (Abbott), are being used, evaluated or developed for use on drug-eluting stents.

Because there is more than one accepted convention for numbering the positions of rapamycin, and derivatives thereof, the numbering convention used herein is depicted below: For reference, the R group for a number of compounds is set forth in the following table:

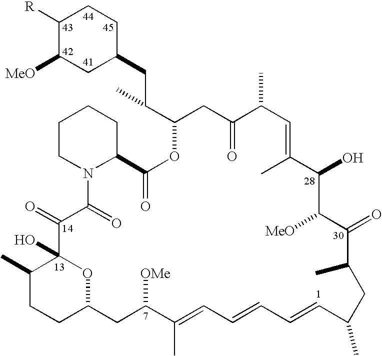

| Compound | —R |
|---|---|
| Rapamycin | —OH |
| AP23573 | —OP(O)(Me)₂ |
| Temsirolimus | —OC(O)C(CH₃)(CH₂OH) |
| Everolimus | —OCH₂CH₂OH |
| Biolimus | —OCH₂CH₂OEt |
| ABT-578 | -Tetrazole |

These compounds are non-limiting examples of potent mTOR inhibitors. For additional information on AP23573, see U.S. Pat. No. 7,091,213. For recent references on temserolimus (CCI779), see WO 2004/026280, WO 2005/011688, WO 2005/070393, WO 2006/086172 and WO 2006/089312. For everolimus, see U.S. Pat. No. 6,384,046, U.S. Pat. No. 6,197,781, U.S. Pat. No. 6,004,973 and WO 2002/066019 and references cited therein. Other mTOR inhibitors of interest include 42-desmethoxy derivatives of rapamycin and its various analogs, as disclosed, e.g., in WO 2006/095185 (in which such compounds are referred to as “39-desmethoxy” compounds based on their numbering system). The derivatives of rapamycin are of particular current interest in practicing this invention.

### 2. Second Drugs

IMiDs refer to the family of thalidomide and its analogs which have immunomodulatory activity. Because of the side effect profile of thalidomide, IMiDs of particular current interest are Revlimid and Actimid (CC-4047).

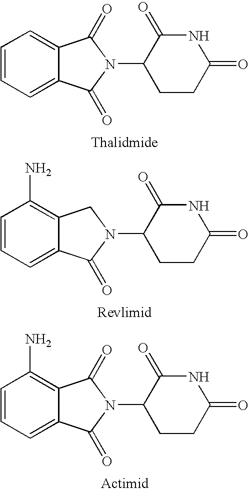

For reference, Revlimid is given orally at a dose of 5-25 mg/daily, depending on the disorder being treated and the patient's tolerability to the drug. Thalidomide has been studied at levels of 100-400 mg/day (orally) and Actimid has been studied at levels of 1-2 mg/day (orally).

PDE4 inhibitors of particular interest include ONO 6126, GSK 842470 (AWD 1 2-281), rolipram and especially roflumilast, roflumilast N-oxide, and cilomilast (Ariflo™, SB207499), the latter of which has been studied at doses of 5, 10 and 15 mg twice a day, orally. Roflumilast has been studied at 250 and 500 μg/day orally.

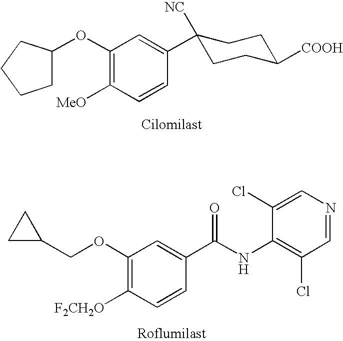

P38 MAP kinase inhibitors of particular interest include BIRB796, SCIO-469 and in particular VX-702, the latter of which has been studied at 5 and 10 mg/day oral dosing. For additional information on p38 inhibitors, see e.g., U.S. Pat. No. 6,608,060, WO9900357A1, WO9958502A1, U.S. Pat. No. 6,800,626, U.S. Pat. No. 6,635,644, WO0170695A1, WO02092087A1 and WO02100405A1.

Xanthine anticytokines include pentoxifylline and a variety of analogs thereof. Illustrative xanthine anticytokines include compounds of the formula:

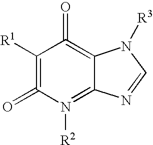

in which R² is a C1-C4 alkyl, and one of R¹ and R³ is of the formula —(CH₂)ₙ-A-CH₃ in which A is (a) a covalent single bond and n is an integer from 0 through 7, or (b) —CO— and n is an integer from 1 through 6, or (c) —C(R⁴)(OH)— where n is an integer from 1 through 6 and R⁴ is H or (C1-C4) alkyl; while the other of R¹ and R³ is H, (C1-C3)alkyl, (C4-C8)cycloalkyl-alkyl or a 1-6 carbon atom alkyl group whose carbon chain is interrupted by an oxygen atom.

Pentoxifylline is the xanthine having the following formula:

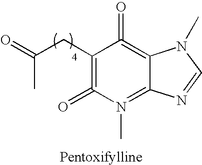

and is available in an extended-release 400 mg tablet which is typically taken 2-3 times a day. For representative background information on xanthine anticytokines, see e.g., U.S. Pat. No. 6,432,968.

The dual TACE/MMP inhibitor of greatest current interest is TMI-1 (see e.g., Pharmacol Exp Ther. 2004 April; 309(1):348-55. Epub 2004 Jan. 12) which has been studied in mice as an oral agent at dose levels of 5, 10, 20 and 100 mg/kg b.i.d.

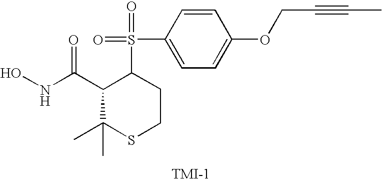

Proteasome inhibitors include compounds such as Velcade. Velcade (bortezomib, also known as PS-341, LDP-341 and MLM-341) has shown activities against a variety of malignancies at intravenous dose level of 0.7 to 1.3 mg/m²/dose biweekly.

A variety of IKK inhibitors are known, including among others, SPC839 and BMS 3455541.

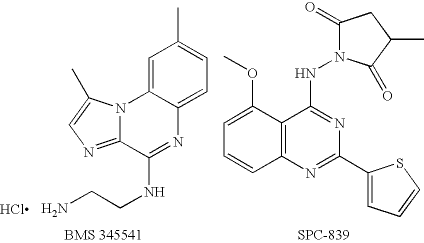

For a recent review, see e.g., Coish et al, “Small molecule inhibitors of IKK kinase activity”, Expert Opin. Ther. Patents (2006) 16(1):1-12. BMS-345541 has been shown to be effective in mouse studies at oral doses of 10-100 mg/kg (see e.g., McIntyre et al, Arthritis Rheum. 2003 September; 48(9):2652-9) while SPC-839 has been shown to have oral efficacy at 30 mg/kg in a rat model.

### 3. Method of Treatments and Prevention

The combination therapy disclosed herein encompasses methods of treating, preventing and/or managing various types of cancer while providing a desirable therapeutic window for achieving clinical benefit without incurring an unacceptable level of side effects.

Examples of cancers and cancer conditions that can be treated with the combination therapy of this document include, but are not limited to, solid tumors such as sarcomas and carcinomas, lymphatic cancers and especially PTEN-deficient tumors (see e.g. Neshat et al, PNAS 98(18):10314 10319; Podsypanina et al, PNAS 98(18):01320-10325; Mills et al PNAS 98(18):10031-10033; Hidalgo et al, Oncogene (2000) 19, 6680-6686). PTEN-deficient tumors may be identified, using genotype analysis and/or in vitro culture and study of biopsied tumor samples. Non-limiting examples of cancers involving abnormalities in the phosphatidyl-inositol 3 kinase/Akt-mTOR pathway include, but are not limited to, glioma, lymphoma and tumors of the lung, bladder, ovary, endometrium, prostate or cervix which are associated with abnormal growth factor receptors (e.g., EGFR, PDGFR, IGF-R and IL-2); ovarian tumors which are associated with abnormalities in P13 kinase; melanoma and tumors of the breast, prostate or endometrium which are associated with abnormalities in PTEN; breast, gastric, ovarian, pancreatic, and prostate cancers associated with abnormalities with Akt; lymphoma, cancers of the breast or bladder and head and neck carcinoma associated with abnormalities in elF-4E; mantle cell lymphoma, breast cancer and head and neck carcinomas associated with abnormalities in Cyclin D; and familial melanoma and pancreas carcinomas associated with abnormalities in P16.

By “solid tumors” are meant tumors and/or metastasis, such as brain and other nervous system tumors (e.g. tumors of the meninges, brain such as glioblastoma and astrocytomas, spinal cord and other parts of the central nervous system); head and/or neck cancer; breast tumors; excretory system tumors (e.g. kidney, renal, pelvis, bladder and other unspecified organs); gastrointestinal tract tumors (e.g. oesophagus, stomach, colon, small intestine, rectum, tumors involving the liver, gall bladder, pancreas and other parts of the digestive organs); oral cavity (lips, tongue, throat, mouth, tonsil, oropharynx, nasopharynx, and other sites); reproductive system tumors (e.g. vulva, cervix, uterus, ovary and other sites associated with female genital organs, penis, prostate, testis and other sites associated with male genital organs); respiratory tract tumors (e.g. nasal cavity, middle ear, sinuses, bronchus, lung and other sites); skeletal system tumors (e.g. bones, cartilage and other sites); skin tumors (e.g. malignant melanoma of the skin, non-melanoma skin cancer, carcinoma, sarcoma); and tumors involving other tissues including peripheral nerves, connective and soft tissue, eye and adnexa, thyroid, adrenal gland and other endocrine glands and related structures, secondary and unspecified malignant neoplasm of lymph nodes, secondary malignant neoplasm of respiratory and digestive systems and secondary malignant neoplasm of other sites. By “lymphatic cancers” are meant e.g. tumors of the blood and lymphatic system (multiple myeloma, lymphoid leukemia, myeloid leukemia, acute or chronic lymphocytic leukemia, monocytic leukemia, other leukemias of specified cell type, leukemia of unspecified cell type, other unspecified malignant neoplasms of lymphoid, haematopoietic and related tissues, for example T-cell lymphoma or cutaneous lymphoma).

Cancers that can be treated using this combination therapy include among others cases which are refractory to treatment with other chemotherapeutics. The term “refractory”, as used herein refers to a cancer (and/or metastases thereof, which shows no or only weak anti-proliferative response (e.g., no or only weak inhibition of tumor growth) after treatment with another chemotherapeutic agent. These are cancers that cannot be treated satisfactorily with other chemotherapeutics. Refractory cancers encompass not only (i) cancers where one or more chemotherapeutics have already failed during treatment of a patient, but also (ii) cancers that can be shown to be refractory by other means, e.g., biopsy and culture in the presence of chemotherapeutics.

The combination therapy described herein is also applicable to the treatment of patients who have not been previously treated for the cancer.

### 4. Formulations, Administration and Use

Formulation of the mTOR Inhibitor:

A variety of oral and parenteral dosage forms are known for rapamycin and a number of rapamycin analogs. Some are currently in use in various treatment methods, monotherapies or otherwise. Those same dosage forms may likewise be used in the practice of the combination therapy disclosed herein. Solid dosage forms are often preferred for oral administration and include among others conventional admixtures, solid dispersions and nanoparticles, typically in tablet, capsule, caplet, gel cap or other solid or partially solid form. Such formulations may optionally contain an enteric coating. Numerous materials and methods for such oral formulations are well known. A typical example of the use of conventional materials and methods to formulate an mTOR inhibitor is shown in US Patent Application US 2004/0077677 and Published International Patent Application WO04026280 (CCl-779). See also U.S. Pat. No. 6,197,781, U.S. Pat. No. 6,589,536, U.S. Pat. No. 6,555,132, U.S. Pat. No. 5,985,321, U.S. Pat. No. 6,565,859 and U.S. Pat. No. 5,932,243.

In a preferred embodiment, the mTOR inhibitor is provided as an oral dosage form, such as a tablet. In the case of AP23573, for instance, the drug may prepared by a wet granulation process. The tablet may contain one or more cellulose polymers and one or more of an antioxidant, chelating agent, filler, binder, surfactant, disintegrant, lubricant, pH-modifying agent and the like. The wet granulation process may be performed with an aqueous or alcoholic, e.g., ethanol, solvent system. Other suitable alcohols include methanol, isopropanol, and the like. The solvent can also be a mixture of solvents, e.g. an alcoholic solvent and water.

It is currently of particular interest that the composition contain from 1 to 45%, from 2 to 35%, from 5 to 25%, or from 8 to 15% by weight of AP23573; from 1 to 50%, from 1 to 35%, from 1 to 15%, or from 2 to 15% by weight of cellulose polymer and from 0.01% to 3%, from 0.05% to 1% or from 0.05% to 0.5% by weight of antioxidant. However, various embodiments may contain more, or less, of these components.

Acceptable antioxidants include, but are not limited to, citric acid, d,l-α-tocopherol, BHA, BHT, monothioglycerol, ascorbic acid, and propyl gallate. It is expected that the antioxidants of the formulations of this invention will be used in concentrations ranging from 0.001% to 3% wt/wt.

Chelating agents, and other materials capable of binding metal ions, such as ethylene diamine tetra acetic acid (EDTA) and its salts are capable of enhancing the stability of AP23573.

Typical cellulose polymers include, but are not limited to hydroxypropylmethylcellulose (HPMC), hydroxypropylmethyl cellulose phthalate, methyl cellulose (MC), hydroxyethyl cellulose, and hydroxypropyl cellulose (HPC).

Acceptable pH modifying agents include, but are not limited to citric acid, sodium citrate, dilute HCl, and other mild acids or bases capable of buffering a solution containing AP23573 to a pH in the range of about 4 to about 6. If present in the composition, the pH modifying agent is usually in amount of up to 1%.

Surfactants may be present in the formulation and include polysorbate 80, sodium lauryl sulfate, sodium dodecyl sulfate, salts of bile acids (taurocholate, glycocholate, cholate, deoxycholate, etc.) which may be combined with lecithin. Alternatively, ethoxylated vegetable oils, such as Cremophor EL, vitamin E tocopherol propylene glycol succinate (Vitamin E TGPS), polyoxyethylene-polyoxypropylene block copolymers, and poloxamers. If present in the composition, the surfactant is usually in amount of up to 20%, for example 1 to 15% by weight.

Binders, fillers, and disintegrants such as sucrose, lactose, microcrystalline cellulose, croscarmellose sodium, magnesium stearate, gum acacia, cholesterol, tragacanth, stearic acid, gelatin, casein, lecithin (phosphatides), carboxymethylcellulose calcium, carboxymethylcellulose sodium, methylcellulose, hydroxyethylcellulose, hydroxypropylcellulose, hydroxypropylmethyl-cellulose phthalate, noncrystalline cellulose, polyvinylpyrrolidone, cetostearyl alcohol, cetyl alcohol, cetyl esters wax, dextrates, dextrin, cyclodextrin, lactose, dextrose, glyceryl monooleate, glyceryl monostearate, glyceryl palmitostearate, polyoxyethylene alkyl ethers, polyethylene glycols, polyoxyethylene castor oil derivatives, polyoxyethylene stearates, and polyvinyl alcohol, and the like may also be incorporated into the formulation.

Any given formulation of this invention may contain multiple ingredients of each class of component. For example, a formulation containing an antioxidant may contain one or more antioxidants as the antioxidant component.

The tablet may further comprise a film-coat to control the release of the rapamycin analog. The tablet may be coated with a film-coat by spraying, dipping or by deposition. The film-coat typically includes a polymeric film-forming material such as copovidone (i.e a copolymer of polyvinylpyrrolidone and vinyl acetate), hydroxypropyl methylcellulose, hydroxypropylcellulose, and acrylate or methacrylate copolymers. Besides a film-forming polymer, the film-coat may further comprise a plasticizer, e.g. polyethylene glycol, triethyl citrate, a surfactant, e.g. a Tween.RTM type, an anti-foaming agent, e.g. Simethicone, and optionally a pigment, e.g. titanium dioxide or iron oxides. The film-coating may also comprise talc as anti-adhesive. The film coat usually accounts for less than about 5% by weight of the dosage form. In a preferred embodiment, the film-coating material comprises copovidone.

The film coating may also be an enteric layer comprising an enteric polymer, for delayed release of the rapamycin analog. An enteric layer is a coating of a substance (i.e a polymer) which is insoluble in the acid medium of the stomach but which is soluble at the higher pH encountered in the intestine. Such materials are used as film coatings on tablets to modify the release of a drug. Suitable enteric polymers are well known to those of skill in the art (WO 01/051031) and include, without limitation, methyl metacrylate polymers, methacrylic acid co-polymers, cellulose acetate phthalate, polyvinyacetate phthalate, hydroxypropyl methyl phthalate, and hydroxypropyl methyl cellulose phthalate. For instance, the enteric layer may comprise a methacrylic acid co-polymer such as Eudragit L100, Acryl-EZE or the like.

In addition to the foregoing non limiting examples of formulation technology, a wide variety of other methods and materials are also well known to those working in the field of macrolides like rapamycin and its derivatives. For additional background and examples of appropriate formulation technologies, see e.g., WO 03/064383 and US Published Patent Application 20050032825.

### Formulation of the “Second” Drug:

The composition containing the second drug for use in the disclosed method, can be prepared in any known or otherwise effective form suitable for use in providing topical or local delivery of the second drug to the affected mucosa.

In certain embodiments, the composition is administered orally in a form permitting it to contact and in some cases to coat, preferably rapidly, the oral and/or esophageal mucosa, to thus provide more effective contact with the affected mucosal tissue. Suitable formulations for topical administration to oral mucosa include liquid formulations (e.g., for mouth rinse, gargle, swish, mouthwash, spray, etc.), solid dosage forms which dissolve in the mouth, and semisolid dosage forms which are applied to coat oral surfaces. Dosage or product forms of this sort include mouthwashes which the individual may swish and swallow or swish and spit out. Suitable dosage forms also include oral lozenges, tablets, gels, and other forms described herein.

Compositions and methods of the present invention can be practiced with any pharmaceutically acceptable product form, usually liquid, that can directly or indirectly affect those areas of mucosa which have become or will likely develop one or more lesions following administration of an mTOR inhibitor.

Suitable compositions may be prepared by any known or otherwise effective method for formulating or manufacturing the selected product form. For example, the second drug can be formulated along with common excipients, diluents, or carriers, and formed into oral tablets, capsules, sprays, mouth washes, mouth washes, swishes, lozenges, treated substrates (e.g., oral or topical swabs, pads, or disposable, non-digestible substrate treated with the compositions of the present invention); oral liquids (e.g., suspensions, solutions, emulsions), powders, or any other suitable dosage form for topical administration to oral mucosa.

In certain embodiments, the second drug is formulated as an elixir, solution, emulsion or suspension for convenient topical oral administration.

Such compositions further include pharmaceutical dosage forms such as lozenges, troches, lollipops or pastilles. These are typically discoid or otherwise shaped solids containing the active ingredient in a suitably flavored base. The base may be a hard sugar or sugar-free candy, glycerinated gelatin, or the combination of sugar with sufficient mucilage to give it form. Troches are placed in the mouth where they slowly dissolve, liberating the active ingredient for direct contact with the affected mucosa.

Troche embodiments are prepared, for example, by adding water slowly to a mixture of the powdered active, powdered sugar, and a gum until a pliable mass is formed. A 7% acacia powder can be used to provide sufficient adhesiveness to the mass. The mass is rolled out and the troche pieces cut from the flattened mass, or the mass can be rolled into a cylinder and divided. Each cut or divided piece is shaped and allowed to dry, to thus form the troche dosage form.

When the second drug is heat stable, or can be rendered heat stable by the use of appropriate processing precautions, it may be prepared in the form of a hard candy base. For example, sugar containing syrup can be concentrated to the point where it becomes a pliable mass. The active ingredient is then added to the mass, which is then kneaded while warm to form a homogeneous mass.

The homogeneous mass is gradually worked into a pipe form having the diameter desired for the candy piece. Lozenges can be cut or sectioned off from the pipe and allowed to cool.

When the second drug is heat labile, it may be made into a lozenge preparation by compression. For example, the granulation step in the preparation is performed in a manner similar to that used for any compressed tablet. The lozenge is made using heavy compression equipment to give a tablet that is harder than usual as it is desirable for the dosage form to dissolve or disintegrate slowly in the mouth. Ingredients are preferably selected to promote slow-dissolving characteristics.

Alternative manufacturing approaches may, of course, be used for any of the various formulations.

In certain embodiments, the second drug is provided in a form which is topically administered to the oral mucosa and then either swallowed or spit out. Formulation types suitable for this route of administration include liquids applied as mouth rinses; solid dosage forms that may dissolve in the mouth; and semisolids that may be applied to oral cavity surfaces.

Stability of the various second drugs varies with structure. However, solids for re-constitution as aqueous based solutions or suspensions prepared either by the patient or by a pharmacist prior to administration to the patient can be used, even for the less stable members.

In some cases the stability of a compound in aqueous solution is pH dependent. Procedures for choosing the optimum pH and buffering agents are well known. Other factors that affect stability in solution are also well known. For example, antioxidants may be added to reduce the rate of degradation due to oxidation.

While the second drug may be in solution or suspension in a simple aqueous system, e.g., in a solution of sodium bicarbonate, the liquid preparation may further contain additional ingredients such as buffers (as mentioned above), surfactants, humectants, preservatives, flavorings, stabilizers (including antioxidants), colorants, and other additives used in preparations administered into the oral cavity.

In certain embodiments, compositions used as mouthwashes have a pH of about 3.5 to about 8. A pH in the range of 4 to 6.5 is also contemplated. Compositions having a pH below 3.5 to 4 can cause a stinging sensation and preparations above pH 6.5 to 8 can be unpleasant to use. However, such unpleasant characteristics can be masked or otherwise made less troublesome for administration using conventional materials and methods.

The preparations are buffered as necessary to provide the appropriate pH. Appropriate buffer systems can include, among others, citrate, acetate, tromethamine and benzoate systems. However, any buffer system commonly used for preparing medicinal compositions would be appropriate. Suitable vehicles include water, alcohols, glycols (polyethylene glycol or polypropylene glycol are examples), glycerin, and the like which are used to solubilize or suspend the active agent(s). Such formulations also optionally include surfactants, which include anionic, nonionic, amphoteric and cationic surfactants. Such surfactants are known in the art as appropriate ingredients for mouthwashes.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Liquid formulations may contain additional components to improve the effectiveness of the product. For example, component(s) may be added to increase viscosity to provide improved retention on the surfaces of the oral cavity. Suitable viscosity increasing agents include carboxyalkyl, hydroxyalkyl, and hydroxyalkyl alkyl celluloses, xanthan gum, carageenan, alginates, pectins, guar gum, polyvinylpyrolidone, and gellan gums. High viscosity formulations may cause nausea in chemotherapy and radiation patients and are therefore not preferred. Gellan gums are sometimes preferred as viscosity modifying agents since aqueous solutions containing certain gellan gums may be prepared so that they will experience an increase in viscosity upon contact with electrolytes. Saliva contains electrolytes that may interact with such a gellan containing solution so as to increase its viscosity in situ.

In certain embodiments, compositions containing the second drug are film-forming or otherwise provide a coating effect on oral mucosa. In some cases they are formulated to contain a mucoadhesive polymer, a viscous polymer gel or a hydrogel, by adaptation of the materials and methods of WO 2004/032843. For instance, a formulation can contain a second drug and at least one cationic polymer or a neutral polymer that becomes cationic upon contact with an aqueous medium such as saliva, thus providing a mucoadhesive or gel forming material.

The cationic polymer can be any pharmaceutically acceptable natural or synthetic polymer which has the desired physical or chemical properties to enhance retention in the mouth. Polymers will typically be cationic polymers, mucoadhesive polymers or polymers which form a gel or hydrogel that physically adheres to the mucosa. In certain embodiments, the cationic polymer is a natural polymer such as gelatin or chitosan. Most synthetic polymers including a relatively high number of carboxylic groups will be mucoadhesive. In certain embodiments, compositions of the present invention comprise one or more natural polymers. Exemplary natural polymers include zein, modified zein, casein, gelatin, gluten, chitosan, collagen, polysaccharides such as cellulose, dextrans, polyhyaluronic acid, and alginic acid. In other embodiments, compositions of the present invention comprise one or more synthetic polymers. Exemplary synthetic polymers include poly(vinyl) alcohols, polyacrylamides, polyalkylene glycols, polyalkylene oxides, polyvinyl esters, PVP, alkyl cellulose (ethyl cellulose, methyl cellulose, etc.), hydroxyalkyl cellulose (e.g. hydroxypropyl cellulose, hydroxypropyl methyl cellulose, hydroxybutyl methyl cellulose, etc.), and the like. Other suitable gel or hydro-gel forming polymers are well known to one of ordinary skill in the art. Other suitable polymers are biodegradable. Suitable materials and methods are as disclosed in WO 2004/032843.

In some embodiments, the second drug is present in a composition in the form of a liquid bandage (see, e.g., U.S. Pat. Nos. 5,081,158; 5,166,233; 5,236,713 and 5,885,611). Such compositions can contain, in addition to a second drug of this invention, one or more of propylene glycol, polyvinylpyrrolidone (also known as povidone or PVP), or hyaluronic acid or a salt thereof, and optionally a flavoring agent and/or a local anaesthetic agent. In other embodiments, compositions of the present invention optionally further comprise glycyrrhetinic acid.

In certain embodiments, hyaluronic acid, or salt thereof, is present in weight percentages ranging from about 0.01 to about 5%. In other embodiments, hyaluronic acid, or salt thereof, is present in about 0.1%. According to another embodiment, glycyrrhetinic acid is present in an amount ranging from about 0.01 to 3% by weight. According to yet another embodiment, PVP is present in an amount ranging from about 1% to about 20% by weight.

“Liquid bandage” formulations based on a cellulose gelling agent are also of interest as a a formulation for the second drug. For instance, such a liquid bandage formulation may contain ethyl cellulose from about 8 to about 12% (or greater) by weight in an ethyl alcohol vehicle (ethanol content from about 50 to about 60% by weight). Other vehicle materials can include purified water (about 5 to about 10% by weight) and propylene glycol (about 2 to about 5% by weight). Benzocaine or lidocaine (or the hydrochloride of either) can be included in amounts of about 10 to about 20% by weight as an optional anesthetic compound. Tannic acid may optionally be included as an astringent compound at a concentration of about 1 to about 5% by weight. Salicylic acid may be included an an optional keratolytic agent at a concentration of about 1 to about 5% by weight.

The various formulations may also contain pharmaceutically inactive ingredients as for example, a sweetener such as sodium saccharin (0.1-1% by weight) and a flavorant (0.1-1% by weight) such as mint or menthol flavors. Flavorings used in the mouthrinse art such as peppermint, citrus flavorings, berry flavorings, vanilla, cinnamon, and sweeteners, either natural or artificial, may be used. Flavorings that are known to increase salivary electrolyte concentrations may be added to increase the magnitude of the viscosity change. The increased viscosity will promote retention of the solutions in the oral cavity and provide greater effectiveness due to increased contact time with the affected tissues.

In order to improve patient acceptability, it is desirable to add an appropriate coloring and/or flavoring material. Any pharmaceutically acceptable coloring or flavoring material may be used. Additionally, any of the mouthwash, mouthrinse, liquid bandage and other liquid compositions of this invention may be chilled to a temperature below body temperature, in some cases below room temperature, e.g. between 30° F. and 40° F., before administration to the patient. Some compositions may be frozen, e.g., in the form of an ice-pop, Popsicle or the like.

Compositions of the present invention optionally further comprise one or more other active ingredients such as antibacterials, disinfectants, antifungals, analgesics, emollients, local anesthetics, and the like.

Additional antimicrobial preservatives may be component of the formulation in cases where it is necessary to inhibit microbial growth. Suitable preservatives include, but are not limited to the alkyl parabens, benzoic acid, and benzyl alcohol. The quantity of preservative may be determined by conducting standard antimicrobial preservative effectiveness tests such as that described in the United States Pharmacopoeia.

Suitable solid dosage forms include powders or tablets that are designed for constitution as solutions by dissolution or suspension in a liquid vehicle and include troches, lollipops, pastilles, or lozenges that dissolve slowly in the mouth. For convenience of use, solids designed to be dissolved to prepare a liquid dosage form prior to administration are rapidly dissolving. Technologies to produce rapidly dissolving solids are well known in the art. These include spray-drying, freeze-drying, particle size reduction and optimizing the pH of the dissolution medium.

In other embodiments, the dosage form is a concentrated gel that is optionally diluted, e.g., with water, prior to administration.

Other medicinal agents may be included in the regimen, e.g., in the composition containing the second drug, for purposes of alleviating other undesirable conditions in the mouth. Such agents may include, for example, local anesthetics, anti-infective agents, and emollients. Examples of local anesthetics are lidocaine and a eutectic mixture of lidocaine and prilocaine. Lidocaine may be administered in solution at a concentration of 2%, at a dose of 15 ml, at intervals of not less than three hours. The eutectic mixture is equimolar, administered at a total concentration of up to 5%. Either could be incorporated in an aerosol at similar doses.

The various compositions may include additional ingredients, such as analgesics for pain relief, antibiotics to lower the risk of or to treat infection, and other agents which might help treat mucositis or promote wound healing. Liquid compositions of the invention may further include a thickening or adhesive agent such as a mucosal-adhesive water-soluble polymer or biocompatible reverse-thermal gelation polymer to help prolong the contact of mucosa with drug, alleviate pain and/or avoid infection. In some other embodiments, the composition may include an agent which promotes cell penetration by the drug.

### 5. Doses and Routes of Administration

The second drug according to the method of the present invention may be administered using topical administration in any amount effective for preventing, treating or lessening the severity of mouth sores induced by the administration of an mTOR inhibitor. The exact amount required will vary from subject to subject, depending on the age, and general condition of the subject, the severity of the infection, the particular agent, its formulation, and the like.

For topical administration of the second drug, the dose level of interest is usually 0.3 to 5 times the dose level approved or studied for the respective drug when given systemically or orally. For example, the dose level of interest is from 0.01 to 2000 mg, from 0.05 to 1000 mg, from 0.1 to 500 mg, from 1.0 to 250 mg. Administration may be once or multiple times daily, weekly (or at some other multiple-day interval) or on an intermittent schedule. By way of example, in the case of Revlimid, dose levels can vary from 1 to 200 mg, or from 1.5 to 100 mg, once or multiple times daily, weekly or as often as needed for preventing, treating or lessening the severity of mouth sores induced by the administration of an mTOR inhibitor. In the case where the second drug is Roflumilast, the dose level is in a range of 0.05 to 5 mg, or from 0.05 to 3 mg.

For topical administration of the second drug, e.g. in a mouth wash, mouth rinse, mouth spray, among others, the extended exposure of the drug is at least half a minute, preferably 1 to 5 minutes or longer. Further extended durations of topical exposure may be beneficial, e.g. for 5-10 minutes or for tens of minutes, especially in cases where the formulation doesn't contain an agent for prolonging the topical exposure, e.g. a gelling or adhesive agent.

The mTOR inhibitor according to the method of the present invention may be administered using any amount and any route of administration effective for treating, preventing and/or managing various types of cancers. The exact amount required will also vary from subject to subject, depending on age, and general condition of the subject, the severity of the cancer, the particular mTOR inhibitor, its mode of administration, and the like.

The specific effective dose level of mTOR inhibitor and of the second drug of the invention for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

It will be understood, however, that the total daily usage of the compounds and compositions of the present invention will be decided by the attending physician within the scope of sound medical judgment.

### 6. Additional Drug Combinations

It will also be appreciated that mTOR inhibitor/second drug combinations of the present invention can be employed in further combination with additional therapies (i.e., a treatment according to the present invention can be administered concurrently with, prior to, or subsequently to one or more desired therapeutics or medical procedures). The particular combination of therapies (therapeutics or procedures) to employ in such a further recombination regimen will take into account compatibility of the desired therapeutics and/or procedures and the desired therapeutic effect to be achieved. It will also be appreciated that the therapies employed may achieve a desired effect for the same disorder (for example, the combination therapy of this invention may be administered concurrently with another agent used to treat the same disorder), or they may achieve different effects (e.g. control of any adverse effects).

For example, methods and compositions of the present invention can be employed together with other procedures including surgery, radiotherapy (e.g., gamma-radiation, neutron beam radiotherapy, electron beam radiotherapy, proton therapy, brachytherapy, a systemic radioactive isotopes), endocrine therapy, hyperthermia and cryotherapy.

Alternatively or additionally, methods and compositions of the present invention can be employed together with other agents to attenuate any adverse effects (e.g., statins, pain medication, antiemetics, G-CSF, GM-CSF, etc.), and/or with other approved chemotherapeutic drugs. Such other drugs include but not limited to one or more of the following: an anti-cancer alkylating or intercalating agent (e.g., mechlorethamine, chlorambucil, Cyclophosphamide, Melphalan, and Ifosfamide); antimetabolite (e.g., Methotrexate); purine antagonist or pyrimidine antagonist (e.g., 6-Mercaptopurine, 5-Fluorouracil, Cytarabile, capecitabine and Gemcitabine); spindle poison (e.g., Vinblastine, Vincristine, Vinorelbine and Paclitaxel); podophyllotoxin (e.g., Etoposide, Irinotecan, Topotecan); antibiotic (e.g., Doxorubicin, Bleomycin and Mitomycin); nitrosourea (e.g., Carmustine, Lomustine); inorganic ion (e.g., Cisplatin, Carboplatin, Oxaliplatin or oxiplatin); enzyme (e.g., Asparaginase); hormone (e.g., Tamoxifen, Leuprolide, Flutamide and Megestrol); proteasome inhibitor (such as Velcade, another proteasome inhibitor (see e.g., WO 02/096933) or another NF-kB inhibitor, including, e.g., an IkK inhibitor); other kinase inhibitors (e.g., an inhibitor of Src, BRC/Abl, kdr, flt3, aurora-2, glycogen synthase kinase 3 (“GSK-3”), EGF-R kinase (e.g., Iressa, Tarceva, etc.), VEGF-R kinase, PDGF-R kinase, etc); an antibody, soluble receptor or other receptor antagonist against a receptor or hormone implicated in a cancer (including receptors such as EGFR, ErbB2, VEGFR, PDGFR, and IGF-R; and agents such as Herceptin (or other anti-Her2 antibody), Avastin, Erbitux, etc.); etc. For a more comprehensive discussion of updated cancer therapies see, http://www.nci.nih.gov/, a list of the FDA approved oncology drugs at http://www.fda.gov/cder/cancer/druglistframe.htm, and The Merck Manual, Seventeenth Ed. 1999, the entire contents of which are hereby incorporated by reference. Examples of other therapeutic agents include among others, Zyloprim, alemtuzumab, altretamine, amifostine, nastrozole, antibodies against prostate-specific membrane antigen (such as MLN-591, MLN591RL and MLN2704), arsenic trioxide, bexarotene, bleomycin, busulfan, capecitabine, Gliadel Wafer, celecoxib, chlorambucil, cisplatin-epinephrine gel, cladribine, cytarabine liposomal, daunorubicin liposomal, daunorubicin, daunomycin, dexrazoxane, docetaxel, doxorubicin, Elliott's B Solution, epirubicin, estramustine, etoposide phosphate, etoposide, exemestane, fludarabine, 5-FU, fulvestrant, gemcitabine, gemtuzumab-ozogamicin, goserelin acetate, hydroxyurea, idarubicin, idarubicin, Idamycin, ifosfamide, imatinib mesylate, irinotecan (or other topoisomerase inhibitor, including antibodies such as MLN576 (XR11576)), letrozole, leucovorin, leucovorin levamisole, liposomal daunorubicin, melphalan, L-PAM, mesna, methotrexate, methoxsalen, mitomycin C, mitoxantrone, MLN518 or MLN608 (or other inhibitors of the flt-3 receptor tyrosine kinase, PDFG-R or c-kit), itoxantrone, paclitaxel, Pegademase, pentostatin, porfimer sodium, Rituximab (RITUXAN®), talc, tamoxifen, temozolomide, teniposide, VM-26, topotecan, toremifene, 2C4 (or other antibody which interferes with HER2-mediated signaling), tretinoin, ATRA, valrubicin, vinorelbine, or pamidronate, zoledronate or another bisphosphonate.

The combination therapy of this invention can also be employed together with one or more further combinations of cytotoxic agents as part of a treatment regimen, wherein the further combination of cytotoxic agents is selected from: CHOPP (cyclophosphamide, doxorubicin, vincristine, prednisone, and procarbazine); CHOP (cyclophosphamide, doxorubicin, vincristine, and prednisone); COP (cyclophosphamide, vincristine, and prednisone); CAP-BOP (cyclophosphamide, doxorubicin, procarbazine, bleomycin, vincristine, and prednisone); m-BACOD (methotrexate, bleomycin, doxorubicin, cyclophosphamide, vincristine, dexamethasone, and leucovorin); ProMACE-MOPP (prednisone, methotrexate, doxorubicin, cyclophosphamide, etoposide, leucovorin, mechlorethamine, vincristine, prednisone, and procarbazine); ProMACE-CytaBOM (prednisone, methotrexate, doxorubicin, cyclophosphamide, etoposide, leucovorin, cytarabine, bleomycin, and vincristine); MACOP-B (methotrexate, doxorubicin, cyclophosphamide, vincristine, prednisone, bleomycin, and leucovorin); MOPP (mechlorethamine, vincristine, prednisone, and procarbazine); ABVD (adriamycin/doxorubicin, bleomycin, vinblastine, and dacarbazine); MOPP (mechlorethamine, vincristine, prednisone and procarbazine) alternating with ABV (adriamycin/doxorubicin, bleomycin, and vinblastine); MOPP (mechlorethamine, vincristine, prednisone, and procarbazine) alternating with ABVD (adriamycin/doxorubicin, bleomycin, vinblastine, and dacarbazine); ChIVPP (chlorambucil, vinblastine, procarbazine, and prednisone); IMVP-16 (ifosfamide, methotrexate, and etoposide); MIME (methyl-gag, ifosfamide, methotrexate, and etoposide); DHAP (dexamethasone, high-dose cytarabine, and cisplatin); ESHAP (etoposide, methylprednisolone, high-dose cytarabine, and cisplatin); CEPP(B) (cyclophosphamide, etoposide, procarbazine, prednisone, and bleomycin); CAMP (lomustine, mitoxantrone, cytarabine, and prednisone); CVP-1 (cyclophosphamide, vincristine, and prednisone), ESHOP (etoposide, methylprednisolone, high-dose cytarabine, vincristine and cisplatin); EPOCH (etoposide, vincristine, and doxorubicin for 96 hours with bolus doses of cyclophosphamide and oral prednisone), ICE (ifosfamide, cyclophosphamide, and etoposide), CEPP(B) (cyclophosphamide, etoposide, procarbazine, prednisone, and bleomycin), CHOP-B (cyclophosphamide, doxorubicin, vincristine, prednisone, and bleomycin), CEPP-B (cyclophosphamide, etoposide, procarbazine, and bleomycin), and P/DOCE (epirubicin or doxorubicin, vincristine, cyclophosphamide, and prednisone).

The following examples contain additional information, exemplification and guidance which can be adapted to the practice of this invention in its various embodiments and the equivalents thereof. The examples are intended to help illustrate the invention, and are not intended to, nor should they be construed to, limit its scope in any way. Indeed, various modification of the invention, and many further embodiments thereof, in addition to those shown and described herein, will be apparent to those skilled in the art upon review of this document, including the examples which follow and the references to the scientific and patent literature cited herein. Such modifications and variations, including design choices in selecting, preparing, formulating and administering the mTOR inhibitor or the second drug of this invention, etc. are intended to be encompassed by the scope of the invention and of the appended claims.

The various pharmaceutical compositions of this invention may be prepared by any known or otherwise effective technique, suitable for making and formulating pharmaceutical dosage forms. Many such methods are described in the pharmaceutical arts or are otherwise well known to those skilled in their respective formulation arts.

### EXAMPLES

### Example 1

### Formulation 01 for Local/Topical Delivery

The formulations may be prepared as a liquid, semi-solid, or solid containing an amount of the second drug of the invention that is effective to treat or prevent mouth sores. Generally, these compositions contain about 0.001 to 800 mg/mL, 0.01 to 500 mg/mL, 0.5 to 500 mg/mL, 10 to 400 mg/mL, 25 to 300 mg/mL, 25 to 250 mg/mL or 25 to 100 mg/mL of the compound. The effective amount of the second drug will depend upon the potency of the compound chosen, the nature of the vehicle and excipients, and the frequency of administration. By way of example, in the case of pentoxifylline, these compositions generally contain a relatively higher dosage, in a range of about 100 to 800 mg/mL of the compound. In the case where the second drug is Revlimid, these compositions generally contain from 0.5 to 200 mg/mL or from 1 to 200 mg/mL of the compound. In the case where the second drug is Roflumilast, these compositions generally contain 0.005 to 10 mg/mL, e.g., 0.01 mg to 10 mg/mL or 0.5 to 5 mg/mL, of the compound.

A. Buffered Solutions

Solutions of the second drug can be prepared by mixing the compound to a concentration of 0.001 to 800 mg/mL with a solution containing methyl and propyl parabens as antimicrobial preservatives at concentrations of 0.18% and 0.02%, respectively and tromethamine buffer.

B. Gellan Gum Buffered Formulations.

Formulations of the second drug can be prepared by adding the compound to a solution containing gellan gum at a concentration of 0.5 mg/mL. The concentration of the second drug can vary between 0.001 to 800 mg/ml depending on the specific chosen compound. The solution also contains methyl and propyl parabens as antimicrobial preservatives at concentrations of 0.18% and 0.02%, respectively and tromethamine buffer.

C. Lower Strength Gellan Gum Formulations.

Lower strength formulations of the second drug can be prepared by adding the compound to a solution containing gellan gum at a concentration of 0.5 mg/mL. The second drug concentration can vary between 0.001 to 800 mg/mL. The solution also contains methyl and propyl parabens as antimicrobial preservatives at concentrations of 0.18% and 0.02%, respectively and tromethamine buffer.

D. Oral Rinses.

A mixture of a selected second drug and a buffer to promote rapid dissolution is subjected to granulation. The resultant material is dissolved in water to form an oral rinse solution containing 0.001 to 800 mg of the compound/mL of formulation.

E. Effervescent Tablets.

Effervescent tablets can be prepared by mixing a selected second with sodium bicarbonate. The tablet is then dissolved in water to form an oral rinse solution containing 0.001 to 800 mg/mL of compound.

F. Rapidly Disintegrating Formulations

Rapidly disintegrating formulations of the second drug may also be prepared as described in WO 2004/000223, substituting the second drug in accordance with this invention for the tetracycline compound disclosed there.

### Example 2

### Formulation 02

In another approach, the second drug is formulated with a biocompatible reverse-thermal gelation polymer using the materials and methods of WO 02/41837.

### Example 3

### Formulation 03

In another approach, the second drug is administered in a concentrated oral gel formulation. In this case, the second drug in an amount from 0.001 to 800 mg, usually 0.1-500 mg of the compound is combined with the contents of one packet of Gelclair™ (OSI Pharmaceuticals) and one tablespoon of water and stirred well. The mixture is used to rinse the mouth for at least 1 minute or as long as possible to coat the tongue, palate, throat, inside of cheeks and all oral tissue well. The material is gargled and then spit out, and administration is repeated 3 times per day or as needed, all in accordance with the normal directions for use of the Gelclair product.

### Example 4

### Formulation 04

In another approach, the second drug of the invention can be stirred into a glass of water to which a dose of AlkaSelzer is added and the mixture is swished and spit out or swished and ingested, in accordance with the normal directions for the use of alka selzer.

### Example 5

### Formulation of a Rapamycin Analog AP23573

The following procedure was used to prepare a tablet containing 10 mg of AP23573 and containing the following components. The tablets are coated with two different coatings—a film-coated tablet for immediate release and an enteric-coated tablet for delayed release. The composition of the core tablet is shown in the following table. Core tablets are film-coated and may be used as such, or may be enteric-coated.

| Component | Weight Percent |
|---|---|
| AP23573 | 8.00% |
| Butylated Hydroxytoluene | 0.08% |
| Hydroxy Propyl Cellulose | 8% |
| Lactose Monohydrate | 50.57% |
| Microcrystalline Cellulose | 30.85% |
| Croscarmellose Sodium | 2.00% |
| Magnesium Stereate | 0.50% |
| Dehydrated Alcohol (Ethanol)* | — |

*Use in processing but does not appear in final product

Hydroxypropyl Cellulose, Lactose Monohydrate, Microcrystalline Cellulose, and half of the Croscarmellose Sodium, were mixed in a high shear granulator. The AP23573 and Butylated Hydroxytoluene (BHT) were dissolved in Dehydrated Alcohol, USP, mixing not less than 45 minutes. The solution of AP23573 and BHT was added to the granulator and mixed to a wet mass for approximately 3 minutes.

The granulation was dried in a fluid bed dryer at 45-55° C. for 60-90 minutes, after which the dried granulation was passed through a mill fitted with a 0.045-inch screen opening to remove oversized granulation. The milled granulation was then blended with Magnesium Stearate, NF and the remaining half of the Croscarmellose Sodium, NF.

The granulation was pressed into tablets using a tablet press set up with 6 mm round concave tooling. The press was adjusted as required for a target tablet weight of 125.0 mg, hardness of 5.5 kp, friability no more than 1%, and disintegration time less than 10 minutes.

### Film Coating

A film coating may be prepared according to following procedure using the following components. The tablets are added to a coating pan and are coated with a solution of Copovidone in Dehydrated Alcohol, USP (20:80 w/w), maintaining a product temperature of 20-35° C., until a weight gain of 5% is achieved. The pan is then cooled and the film-coated tablets allowed to dry. Film-coated tablets may be packaged as such, or may be enteric coated.

### Enteric Coating

An enteric coating may be prepared according to following procedure using the following components.

| Film Coating | Percent of Suspension |
|---|---|
| Methacrylic Acid Copolymer | 11.03% |
| Triethyl Citrate | 2.16% |
| Talc | 2.81% |
| Dehydrated Alcohol (Ethanol)* | 84.00% |

*Use in processing but not for retention in final product

For enteric coating, the tablets are placed in a coating pan and coated with a suspension of Methacrylic Acid Copolymer, NF, Triethyl Citrate, NF, and Talc in Dehydrated Alcohol, USP, maintaining a product temperature of 20-35° C., until a weight gain of 8% is achieved. The pan is then cooled, and the enteric-coated tablets allowed to dry.

### Example 6

### Methods of Treatment

1. Methods of using these formulations generally involve applying the formulations topically to mucosal surfaces of the oral cavity and gastrointestinal tract. One to six or more applications are made per day beginning at least 24 hours (i.e: 1, 2, 3 days or 1 week) before the administration of the mTOR inhibitor; the topical administration of the second drug is continued thereafter until conclusion of treatment or as needed thereafter to prevent, treat or alleviate mouth sores.
The typical volume of a mouthwash would be between 5-15 ml of a composition containing 0.001-80 mg of the second drug of this invention.
2. Topical administration of a second drug according to previously described formulations in combination with AP23573 in the treatment of refractory or advanced cancers
One day to one week after the first topical administration of a second drug of this invention, the patient begins a repeating cycle of daily dosing of AP23573 on one of the following schedules:
(a) 30 mg of AP23573 orally every day for six consecutive days, followed by 1, 8, 15 or 22 days without additional AP23573, then starting the next cycle of treatment with AP23573.
(b) 30 mg of AP23573 orally every day for five consecutive days, followed by 2, 9, 16 or 23 days without additional AP23573, then starting the next cycle of treatment with AP23573.
(c) 10-40 mg of AP23573 orally every day for 28 days, then starting the next cycle without interruption of treatment.
(d) 10-40 mg of AP25373 orally every day for 21 days, followed by 7 days without additional AP23573, then starting the next cycle of treatment with AP23573.
(e) 40-80 mg of AP23573 administered orally in any of the schedules (a) through (d).
3. Topical administration of a second drug according to previously described formulations in combination with intravenous AP23573 in the treatment of refractory or advanced cancers
One day to one week after the first topical administration of a second drug of this invention, the patient begins a repeating cycle of daily dosing of AP23573 on one of the following schedules:
(a) 10-40 mg of AP23573 injected every day for 5 consecutive days, followed by 2 or 9 days without additional AP23573, then starting the next cycle of treatment with AP23573.
(b) 40-80 mg of AP25373 injected every day for 5 consecutive days, followed by 2 or 9 days without additional AP23573, then starting the next cycle of treatment with AP23573.
4. Topical administration of a second drug according to previously described formulations in combination with oral Temsirolimus in the treatment of refractory or advanced cancers
One day to one week after the first topical administration of a second drug of this invention, the patient begins a repeating cycle of daily dosing of Temsirolimus on one of the following schedules:
(a) 20 mg of temsirolimus orally every day for six consecutive days, followed by 1, 8, 15 or 22 days without additional temsirolimus, then starting the next cycle of treatment with temsirolimus.
(b) 30-80 mg of temsirolimus orally every day for six consecutive days, followed by 1, 8, 15 or 22 days without additional temsirolimus, then starting the next cycle of treatment with temsirolimus.
(c) any of the previous doses in schedules (a) or (b), where Temsirolimus is administered orally every day for five consecutive days, followed by 2, 9, 16 or 23 days without additional Temsirolimus, then starting the next cycle of treatment with Temsirolimus.
(d) 30-75 mg of Temsirolimus orally every day for five consecutive days, every 2 weeks.
(e) 30-75 mg of Temsirolimus orally every day for 7 consecutive days, followed by 8 days without additional Temsirolimus, then starting the next cycle of treatment with Temsirolimus.
5. Topical administration of a second drug according to previously described formulations in combination with intravenous Temsirolimus in the treatment of refractory or advanced cancers
One day to one week after the first topical administration of a second drug of this invention, the patient begins a repeating cycle of dosing of Temsirolimus on one of the following schedules:
(a) 75 mg of temsirolimus in a 30 minute intravenous infusion on days 1, 8, 15 and 22 (i.e. day 1 being 2 weeks after the first topical administration of a second drug). In the absence of disease progression and unacceptable toxicity, the cycle repeats every 28 days.
(b) 75-250 mg of temsirolimus in 30 minutes intravenous infusion on days 1, 8, 15 and 22 (i.e. day 1 being 2 weeks after the first topical administration of a second drug). In the absence of disease progression and unacceptable toxicity, the cycle repeats every 28 days.
(c) 250 mg and higher doses of temsirolimus in 30 minutes intravenous infusion on days 1, 8, 15 and 22 (i.e. day 1 being 2 weeks after the first topical administration of a second drug). In the absence of disease progression and unacceptable toxicity, the cycle repeats every 28 days.
6. Topical administration of a second drug according to previously described formulations in combination with Everolimus in the treatment of refractory or advanced cancers
One day to one week after the first topical administration of a second drug of this invention, the patient begins a repeating cycle of daily dosing of Everolimus on one of the following schedules:
(a) 1.5 to 5 mg of Everolimus orally every day every day for five or six consecutive days, followed by 1, 8, 15 or 22 days without additional Everolimus, then starting the next cycle of treatment with Everolimus.
(b) 10-20 mg of Everolimus orally every day for six consecutive days, followed by 1, 8, 15 or 22 days without additional Everolimus, then starting the next cycle of treatment with Everolimus.
(c) 20 mg and higher doses of Everolimus orally every day for six consecutive days, followed by 1, 8, 15 or 22 days without additional Everolimus, then starting the next cycle of treatment with Everolimus.
(d) any of the previous doses in schedules (a) through (c), where Everolimus is administered orally every day for five consecutive days, followed by 2, 9, 16 or 23 days without additional Everolimus, then starting the next cycle of treatment with Everolimus.
(e) any of the previous doses in schedules (a) through (c), where Everolimus is administered orally once daily for 28 days. In the absence of disease progression and unacceptable toxicity, the treatment continues for an additional 28 days and so on.

1. A method for treating a cancer patient which comprises administering to the patient an mTOR inhibitor and a second drug selected from the following: an IMiD, a PDE4 inhibitor, a p38 MAP kinase inhibitor, a xanthine anticytokine, a dual TACE/MMP inhibitor, a proteasome inhibitor or an IKK inhibitor; wherein the second drug is administered locally to the oral mucosa.

2. The method of claim 1 wherein the second drug is thalidomide, Revlimid, Actimid, SB207499, BIRB 796, SCIO-469, VX-702, pentoxiphylline, TMI 1, BMS 345541 or Velcade.

3. The method of claim 1, wherein the mTOR inhibitor is administered orally or parenterally.

4. The method of claim 2, wherein the mTOR inhibitor is administered orally or parenterally.

5. The method of claim 1, wherein the mTOR inhibitor is administered at an average cumulative weekly dose level of 25 to 300 mg.

6. The method of claim 2, wherein the mTOR inhibitor is administered at an average cumulative weekly dose level of 25 to 300 mg.

7. The method of claim 1, wherein the mTOR inhibitor is administered at an average daily dose level of 5 to 60 mg.

8. The method of claim 2, wherein the mTOR inhibitor is administered at an average daily dose level of 5 to 60 mg.

9. The method of claim 1 wherein administration of the second drug is begun at least 1 day prior to the first administration of the mTOR inhibitor.

10. The method of claim 2 wherein administration of the second drug is begun at least 1 day prior to the first administration of the mTOR inhibitor.

11. The method of claim 9, wherein administration of the second drug is begun at least 1 week prior to the first administration of the mTOR inhibitor.

12. The method of claim 10, wherein administration of the second drug is begun at least 1 week prior to the first administration of the mTOR inhibitor.

13. The method of claim 9, wherein administration of the second drug is initiated 7-21 days prior to the first administration of the first drug.

14. The method of claim 10, wherein administration of the second drug is initiated 7-21 days prior to the first administration of the first drug.

15. The method of any of claims 1-14, wherein the cancer is a sarcoma, lymphoma, or leukemia or a cancer of the bladder, colon, brain, breast, head and neck, endometrium, lung, ovary, pancreas or prostate.

16. The method of claim 15, wherein one or more additional drugs are administered to the patient, selected from a statin, an analgesic, or G-CSF.
